(19) United States
(12) Patent Application Publication
(10) Pub. No.: US 2006/0111721 A1
(43) Pub. Date: May 25, 2006

(54) Staple for the suture of the sternum

(76) Inventors: Cesare Puricelli, Fagagna (IT); Gianni Ponte, Udine (IT)

Correspondence Address: Abelman Frayne & Schwab, 150 East 42nd Street, New York, NY, 10017-5612, US

(21) Appl. No.: 10/524,453

(22) PCT Filed: Aug. 8, 2003
(86) PCT No.: PCT/EP03/08821

§ 371 Date: Aug. 31, 2005

(30) Foreign Application Priority Data
Aug. 9, 2002 (IT) MI2002A001817

(51) Int. Cl. A61F 2/30

(52) U.S. Cl. 606/75

(57) ABSTRACT

A staple for the suture of the sternum having a central body with right and left threaded ends in which two teeth are screwed able to be introduced into suitable holes made astride a median sternotomy performed on a patient. Rotating the central body draws the teeth together or apart, thus tightening the two halves of the sternum with appropriate tension. The staple is suitable to be tightened by means of an apparatus provided with friction, to give the correct tightening tension. Also described are a method for the suture of the sternum and punch cutter forceps suitable to be used in such operation.

### FIELD OF THE INVENTION

The present invention concerns a staple for the suture of the sternum; it is of particular use after operations in which a sternotomy proved necessary, such as in operations of cardiosurgery.

The invention also concerns a method of application of the staple and a kit including one or more staples.

The invention also concerns punch cutter forceps to make holes which are able to receive a staple.

### PREVIOUS TECHNIQUE

Currently, the sternotomy represents the most commonly practiced system of access to the thoracic cavity in operations like those of cardiosurgery. Closure of the sternum, at the end of the operation, is commonly performed by passing metal wires around the two halves into which it has been divided; the wires are wound in front of the sternum which are then tightened, thus closing the two parts of the bone. Each wire can be wound once or twice, in the latter case it is wound crosswise. Six wires passing once or three times are commonly used for the complete suture of the sternum. The technique involves various problems. Since closure is carried out manually by the surgeon, generally the tension of the wires is unequal, with reduced closing efficacy, with possible dehiscence (the tightest ligature will tend to cut into the bone, thus working loose. In addition, there is often a certain staggering of the two bone halves which are drawn together.

Moreover, the effort required in passing the wires is considerable, and it requires a sharp needle with the risk of lesions to the detriment of the surgeon. As for the patient, passing the wire around the sternum involves the risk of lesions to the internal thoracic arteries and also to the pleurae and the lungs.

Staples have been proposed made of elastic type alloys to be placed astride the sternum to secure the two halves. They are alloys which are able to expand at low temperatures (with the possibility of positioning the staple astride the sternum) and of regaining their original form at body temperature. These are very expensive, and besides the fact that the tension of the staple cannot be adjusted, thus not solving the problems of unequal tensions in the various staples, the position astride the sternum still involves the risk of lesions to the internal thoracic arteries. Another proposed system envisages the opening of holes, in the sternum, by means of punch cutter forceps, symmetrical with regard to the sternotomy section. In each pair of symmetrical holes is inserted an elastic staple, kept widened by special forceps. Upon releasing the forceps the tension of the staple will be determined by the distance of the holes, which is carefully predetermined. However, a certain irregularity in the positioning of the holes can still produce uneven distribution of the tensions in the staples, and the accuracy required during perforation can complicate and lengthen the operation. Moreover, the staples are manipulated by forceps and inserted under tension, thus making the operation more complicated and less easy, and therefore also potentially injurious.

### SUMMARY

The problems discussed above have now been resolved according to the present invention by means of a new type of staple for the suture of the sternum, including a central body having at least two opposite threaded parts and at least two teeth each having a threaded end which is able to pair up with one of the threaded parts of the central body. According to a preferred aspect of the invention, said central body has an axis of symmetry, more preferably is cylindrical and the two opposite threads (that is a right and a left one) are situated at the two ends of the central body; the latter will be preferably a hollow cylinder, at least in part, and the two threads will be internal threads. The two teeth will have a threaded end and a leg forming a certain angle with the former, preferably an acute angle. The two teeth, obviously threaded one right and one left, will be approximately L-shaped or, better, shaped in the form of a “7”. Rotating the central body will draw the two teeth together or apart, thanks to the opposite threads.

The staple will be preferably made of biocompatible material, more preferably diamagnetic, so as not to interfere with diagnosis and monitoring equipment. For example biocompatible steel or titanium alloys can be used.

### LIST OF THE FIGURES

The present invention will now be illustrated by the detailed description of a preferred, but not exclusive, embodiment, supplied purely by way of example, with the aid of the enclosed figures of which:

FIG. 1 schematically represents a longitudinal section of the central body of a staple according to the present invention;

FIG. 2 schematically represents a tooth of a staple according to the present invention;

FIG. 3 schematically represents the staple according to the present assembled invention;

FIGS. 4a and 4b schematically represent, in front and side section, an apparatus to give the correct tension to the staple;

FIG. 5 schematically represents, in section, punch cutter forceps to make holes in a bone, in particular the sternum.

### DETAILED DESCRIPTION

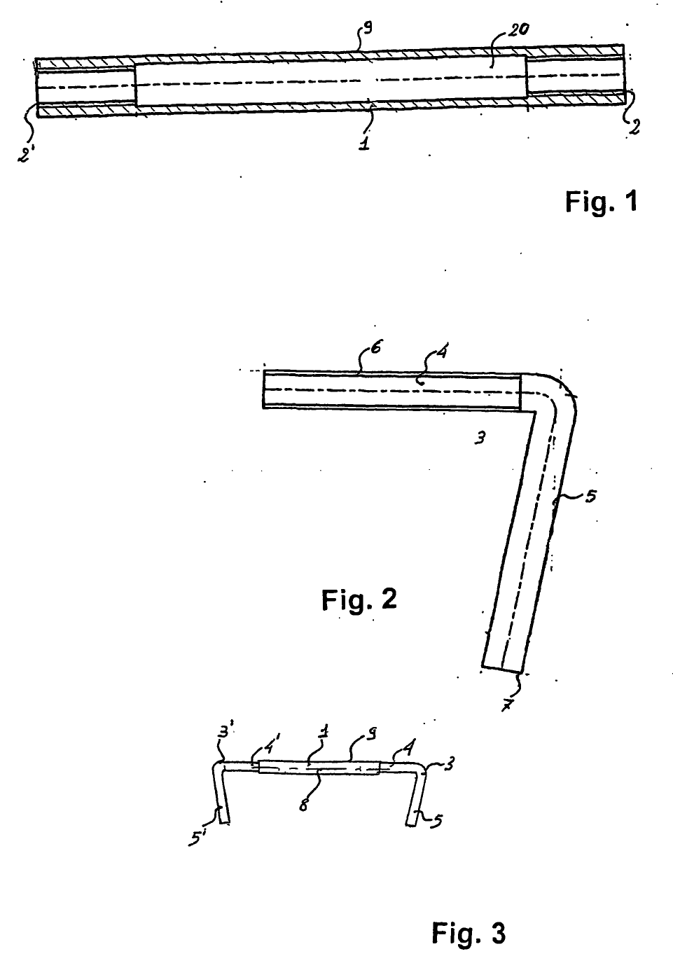

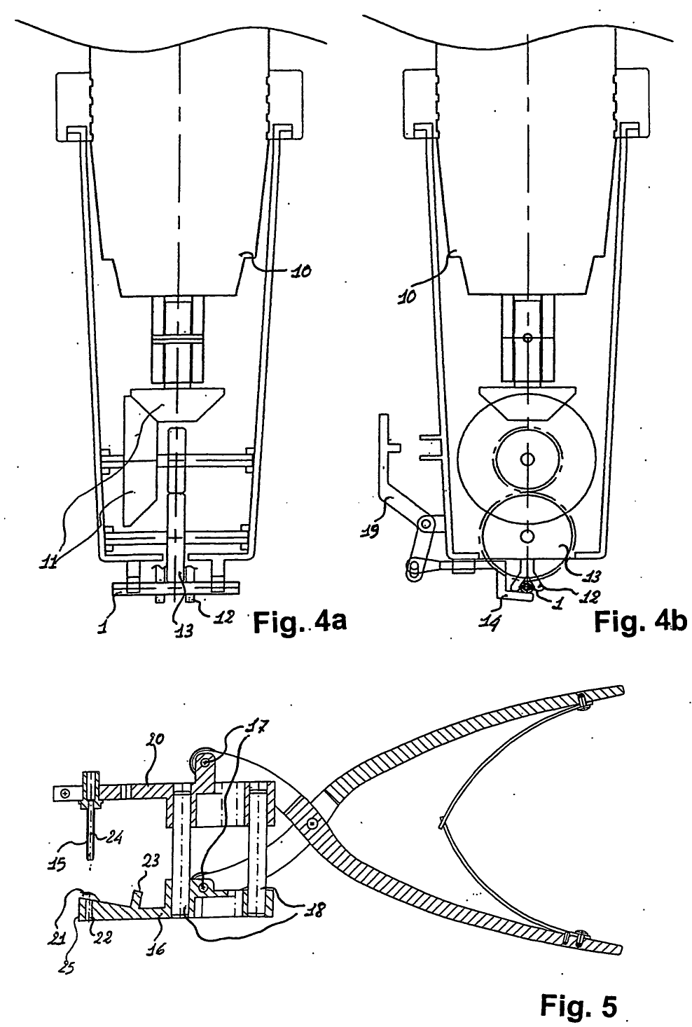

With reference to FIGS. 1, 2 and 3 a staple is now described according to the present invention. The central body 1 in this case is cylindrical and is hollow. At its ends it has the internal threads 2 and 2′, one right and one left. The tooth 3 of FIG. 2 has an end 4 that has the external thread 6, able to be joined to the thread 2 of the central body 1 and a leg 5 that forms a certain angle with the end 4. Preferably the leg 5 will be cylindrical and its axis will form an acute or right angle with the axis of the threaded end 4. For example, this angle can be between 70° and 85°, a preferred value being 80°. The tip 7 of the tooth 3 can have advantageously rounded edges, the tip can even be hemispherical or of other shape capable of not injuring body tissues; in fact it is destined to be introduced into a hole made in the sternum, and is also able to protrude from the hole. The staple also includes another tooth 3′, a mirror image of the tooth 3. When it is assembled (FIG. 3), rotating the central body around its own axis 8 will screw the teeth 3 and 3′ drawing them together. Rotation in the opposite direction will unscrew them. The central body can advantageously be knurled on the external surface 9 or part of it, or have other means to aid grip (manual or a special apparatus). The staple is intended for the suture of a sternum (human or also animal) after sternotomy. Sternotomy involves the division of the sternum into two with longitudinal section. To perform the suture, the legs 5 and 5′ of the teeth of the staple are inserted into holes made in the sternum astride the sternotomy section. By rotating the central body 1 the legs 5 and 5′ are drawn towards each other to give correct tension to the staple. When the legs 5 and 5′ form an acute angle with the ends 4 and 4′ of the teeth 3 and 3′, as indicated above, their inclination will help to prevent the withdrawal of the legs from the holes. In addition, the surface of the legs 5 and 5′ may advantageously show protuberances, suitable grooves to encourage their grip on the bone. According to a possible embodiment the legs can be threaded, and it is also possible to obtain the teeth from wholly threaded-cylindrical pieces which are appropriately bent. The tension of a staple can be given by means of an apparatus like that of FIGS. 4a and 4b. It has a motor unit 10 powered, for example, by battery or rechargeable batteries, unit that through the means 11 is suitable to transmit the motion (wheels and gears) to rotate the central body 1 of a staple held by special means. The wheel 13 is pressed against the body 1 and transmits the motion by friction. The apparatus is equipped with mechanical, electronic or other type of clutch to give the staple a predetermined tension. In this case the means are a pair of supports 12 and a tongue 14 removable by operating the lever 19 after screwing has occurred. Other embodiments of the staple holding system can be envisaged, particularly to eliminate the tongue 14, that can keep the staple pulled aside from the sternum, a staple which must then be pushed for its total insertion.

A method of operating, for use of the staple according to the present invention is as follows.

The sternum is exposed and, for example, with an electrosurgical knife, the line is marked out along which the sternotomy is performed. Pairs of points are marked astride this on which holes are made, generally 5 or 6 pairs. They can be marked by using special stencils, or templates having the points of the holes marked and references to be placed on the line of sternotomy, so as to have exact symmetry of the holes as regards the latter. Otherwise perpendicular lines to that of the sternotomy can be marked with the electrosurgical knife, on which the holes must be performed. It is important that the holes of each pair lie with their centre on one of these lines (so that the tension of the staple acts perpendicularly to the sternotomy and does not exert longitudinal sliding stress on the two hemisterna. The sternotomy is performed, and before or, preferably, after the actual operation, the holes are made in the selected points by using suitable means such as punch cutter forceps, for example like those of FIG. 5, which represent another aspect of the invention. A first jaw 20 carries a punching tip 15, for example a cylinder with sharp edges. Thanks to suitable means, such as the system of hinges and guides 17 and 18, the tip 15, following a straight line, moves closer to the second jaw 16, between which jaw and the tip 15 the bone is compressed and cut, in such a way that a small cylinder thereof is removed. A through hole 22 can be provided on the second opposite jaw 16, to receive the tip 15 and permit cutting. The portion 21 of surface of the second jaw 16 facing the tip 15 can be suitably inclined, with respect to a line perpendicular to the axis 24 of the tip 15 so that by inserting the jaw 16, behind the hemisternum to be pierced and putting the jaw in contact with it, the jaw gives the correct inclination to the tip, in order that the hole is inclined in a manner corresponding to the leg of the staple which it is going to receive. This portion 21 can form an acute angle with the axis 24 of the tip, which is equal to that formed by the leg of a staple tooth with the threaded end of the tooth (when said angle is acute). In order to correctly position the hole (avoiding that it is too far from the sternotomy) a protuberance 23 can be envisaged on the second jaw 16, facing the first jaw 20 at a suitable distance from the end 25 of said jaw.

After the operation is ended, the hemisterna are brought together by means of special approximators, the legs of the teeth of a staple are introduced into a pair of holes and it is tightened to the desired tension as seen above. Unlike that with elastic or expandable type staples, in which the distance between the holes must be precise, since the tension placed on the staple is determined by deformation, the staple according to the invention allows greater freedom thanks to the applicable tensioning system, which allows punctual consideration of the bone characteristics and other operational requirements.

Since the sternum has a spongy inner layer it is expedient that the legs of the teeth of the staple grip on both the more resistant outer layers. For this purpose staples with legs of such a length will be chosen so as to completely cross the sternum, and, if necessary, protrude by a few mm. For example, with the inclinations of the leg given above with respect to the threaded end of the tooth, leg lengths can be comprised between 10 and 20 mm, for they may be for example 15 mm. The teeth can have diameters of between 1 and 5 mm, for example 2 mm, the central body being dimentioned accordingly. It can have a total length of between 10 and 40 mm, for example 20 mm, the distance between the holes to which the staple is adapted varying within a wide range, thank to its adjustability due to the double thread. Dimensions outside the specified ranges, for particular requirements, are also possible.

In the case of FIGS. 1, 2 and 3 item he noted that the cavity 20 has such a diameter to receive part of the threaded ends 4 and 4′ of the teeth. In this way, by appropriately choosing the lengths, the threads 2 and 2′ will be completely screwed, whatever is the operative opening of the staple.

The invention also concerns a surgical kit including one or more staples according to the present invention and an apparatus equipped with clutch suitable to rotate the cylindrical body of a staple whose teeth are held in holes made in a bone, up to a preset tension.

The kit may advantageously include one or more punch cutter forceps able to make sternal holes of a diameter suited to the staples included in the kit.

1. Staple for the suture of a sternum, including a central body (1) having at least two threaded parts (2, 2′) with opposite threads and at least two teeth (3, 3′), each having a threaded end (4, 4′) able to engage one of the threaded parts of the central body.

2. Staple as in claim 1 wherein said central body is a hollow cylinder and the two opposite threads are internal threads situated at the two ends of the central body.

3. Staple as in claim 1 wherein each tooth has one leg (5, 5′), having an axis forming a right or acute angle with the threaded end (4, 4′).

4. Staple as in claim 3 wherein said angle is between 70° and 85°.

5. Staple as in claim 3 wherein the edges of the tip (7) of each leg are rounded.

6. Staple as in claim 1 made of biocompatible and diamagnetic material.

7. Staple as in claim 1 wherein the external surface (9) of the central body is knurled.

8. Staple according to claim 3 wherein the legs of the teeth have a length of between 10 and 20 mm.

9. Punch cutter forceps able to pierce a sternum, including a first jaw (20) having a punching tip (15) having an axis (24), a second jaw (16), means (17, 18) able to move the tip, according to a rectilinear trajectory, closer to the second jaw, a portion (21) of surface of the second jaw forming an acute angle with the axis of the punching tip.

10. Forceps as in claim 9 wherein said angle is between 70° and 85°.

11. Forceps as in claim 9 or 10 wherein on the second jaw (16) there is a protuberance (23) facing the first jaw.

12. Surgical kit including:
one or more staples for the suture of a sternum, said staples including a central body (1) having at least two threaded parts (2, 2′) with opposite threads and at least two teeth (3, 3′), each having a threaded end (4, 4′) able to engage one of the threaded parts of the central body; and
an apparatus equipped with clutch able to rotate the cylindrical body of a staple whose teeth are held in holes made in a bone, up to a preset tension of the staple.

13. Surgical kit as in claim 12 including one or more punch cutter forceps able to make holes, in a sternum, of diameter suitable for one or more staples included in the kit.

14. Surgical kit as in claim 13 including one or more punch cutter forceps, said forceps-including a first jaw (20) having a punching tip (15) having an axis (24), a second jaw (16), means (17, 18) able to move the tip, according to a rectilinear trajectory, closer to the second jaw, a portion (21) of surface of the second jaw forming an acute angle with the axis of the punching tip.
